(19) United States
(12) Patent Application Publication
(10) Pub. No.: US 2026/0263265 A1
(43) Pub. Date: Sep. 10, 2026

(54) AVA COOLING MITTEN

(71) Applicant: Bradford Stoler, Evergreen, CO (US)

(72) Inventor: Bradford Stoler, Evergreen, CO (US)

(21) Appl. No.: 19/070,514

(22) Filed: Mar. 4, 2025

(51) Int. Cl. A61F 7/02; A61F 7/00

(52) U.S. Cl. CPC A61F 7/02; A61F 2007/0036; A61F 2007/0087; A61F 2007/0204; A61F 2007/023; A61F 2007/0233

(57) ABSTRACT

An AVA cooling mitten optimizes core thermoregulation via Arterio-Venous Anastomoses zones to lower core body temperature by sequentially or simultaneously cooling at least one of the AVA zones on the hands (palms, fingers, and wrists) and at least a portion of the facial AVA zones (cheeks and forehead) within an optimal temperature range to induce core body thermoregulation. Embodiments include a mitten that also enables simultaneous cooling of hand and facial AVA areas using a cooling element configured to maintain cold to cool temperatures for a maximum period of time, the cooling element surrounded by an insulating layer configured to transmit optimal AVA temperatures between 45° F. and 59° F. The AVA cooling mitten is affordable, portable, and easy-to-use for athletics, outdoor work, and in extreme or critical situations, such as to prevent heat stroke. An open-end embodiment has a foreshortened hand pocket so fingers can extend beyond the pocket.

### CROSS-REFERENCES TO RELATED APPLICATIONS

This application claims priority from U.S. Provisional Patent Application No. 63/630,785, filed Mar. 4, 2024, herein incorporated by reference in its entirety.

### FIELD OF THE INVENTION

This invention relates generally to cooling the core of a human body, and more particularly to wearable devices for optimized cooling of the core of a human body.

### BACKGROUND OF THE INVENTION

The temperature range that optimizes heat flow through the arteriovenous anastomosis (AVA) zones of the body, specifically those in the human skin, is within the thermoneutral zone. The “optimal AVA temperature range,” to induce heat loss, i.e., cooling, of core body temperature is between 45° F. and 59° F. (optimal temperature range for thermal conductivity via the AVA zones).

In this range, the AVAs, which are abundant in the glabrous skin of the hands and feet, play a significant role in temperature regulation. When the body temperature is within or near the upper end of the optimal temperature range, the AVAs are more likely to be open, facilitating a low-resistance pathway for blood to flow directly from arteries to veins, thereby aiding in heat dissipation. Conversely, near the lower end of the optimal temperature range, these anastomoses tend to be closed, conserving heat by reducing blood flow to the skin surface.

Quickly lowering core body temperature can be useful, especially in situations like heat stroke or other condition of hyperthermia.

In previous attempts to treat hyperthermia, research has shown (Caruso et al., Intermittent Palm Cooling Impact on Resistive Exercise Performance. Int J SportsMed; 36: 814-821 (2015)) that the human body has three Arterio-Venous Anastomoses (AVA) zones that allow for maximum thermal conductivity to reduce core body temperature: the palms of hands, the face (cheeks & forehead), and the bottoms of the feet. The skin type of these AVA zones is referred to as “glabrous” skin.

The discovery of AVA zones was based on a Stanford study (H. Craig Heller et al., Control of Breathing, Circulation, and Temperature, Recovery from Mild Hypothermia can be Accelerated by Mechanically Distending Blood Bessels in the Hand (November 1998)). The researchers in this study realized that heating, and potentially cooling, specific zones of the body produced greater thermoregulatory response than other areas of the body. While this study focused on the mitigation of hypothermia symptoms via heating the hands and forearm to quickly increase core body temperature, the hypothesis of using the AVA zones for cooling was proposed.

In a subsequent Stanford study (H. Craig Heller et. al., Work Volume and Strength Training Responses to Resistive Exercise Improve with Periodic Heat Extraction from the Palm, Journal of Strength and Conditioning Research (September 2012)), this hypothesis was confirmed, and concluded that “palm cooling increased work volume by 40% (vs. 13% with no treatment; n=8, p<0.05). Over 6 weeks of pull-up training, palm cooling increased work volume by 144% in pull-up experienced subjects (vs. 5% over 2 weeks with no treatment)”. These results of 40% increase in bench presses and 144% increase in pull-ups were eye opening!

In another study, the researchers correlated an increase in resistance training performance to cooling core body temperatures (Sub Kwon et. al., Palm Cooling Delays Fatigue During High Intensity Bench Press Exercise, Medicine and Science in Sports and Exercise (February 2020)).

Further, the benefits of AVA cooling have been shown for women (Kwon et. al., Palm Cooling Delays Fatigue During Resistance Exercise in Women, Journal of Strength and Conditioning Research, August 2015).

A wide variety of techniques to achieve optimal AVA cooling temperatures have been tried: fans, water pumps, pressurized air, and electromechanical devices. However, each of these approaches can be expensive, in addition to other drawbacks.

### SUMMARY OF THE INVENTION

A general aspect of the invention is an AVA cooling mitten able to provide efficient cooling to at least one AVA zone of a front side of a wrist and a front side of a hand having a palm and fingers. The AVA cooling mitten includes: an insulating layer configured to form a cooling element pocket, the cooling element pocket configured to receive a cooling element; a first outer layer in thermally-conductive surrounding relationship with the insulating layer; and a second outer layer attached to the first outer layer so as to form a hand pocket, the hand pocket configured to receive and conform to a hand and a wrist, the hand pocket having an upper inside surface and a lower inside surface, the lower inside surface configured to make thermally-conductive contact with AVA zones of the front side of the wrist and the front side of the hand received by the hand pocket.

In some embodiments, the insulation layer is configured to modulate thermal flow from the cooling element so as to maintain the AVA zones of the palm, and the front side of the wrist and fingers at a temperature between 45° F. and 59° F.

In some embodiments, the first outer layer is configured to ensure that the temperature of an AVA zone on a forehead remains within an optimal AVA range of 45° F. to 59° F. when the surface of the first outer layer is pressed against the forehead.

In some embodiments, the cooling element is shaped and sized to be in thermally conducting relationship within the cooling element pocket.

In some embodiments, the cooling element is a re-usable cold pack, and is configured to be frozen before being inserted into the cooling element pocket, and to be removed from the cooling element pocket after the re-usable cold pack melts.

In some embodiments, the cooling element is a non-reusable instant ice-pack that is ready-to-use once inserted into the cooling element pocket.

In some embodiments, the AVA cooling mitten further includes: an attachment device connected to the second outer layer, the attachment device configured to attach to one of: a belt, a strap, a harness, a sock, or a backpack.

In some embodiments, the attachment device is one of: a buckle, a snap, or hook and loop material.

In some embodiments, the re-usable cooling element includes: carboxymethyl cellulose (CMC) or sodium polyacrylate (SAP).

In some embodiments, the non-reusable cooling element includes: water, ammonium nitrate (NH₄NO₃), calcium ammonium, nitrate (can), urea, sodium thiosulfate pentahydrate (Na₂S₂O₃·5H₂O).

In some embodiments, the insulation layer is made using at least one of: fleece, wool, recycled polyester insulation, down, merino wool, aerogel, alpaca fleece.

In some embodiments, the first outer layer is made using at least one of: recycled polyester, neoprene, nitrile, Primaloft®, Thinsulate®, alpaca fleece, Polartec® alpha, aerogel composite materials.

In some embodiments, the second outer layer is made using at least one of: recycled polyester, neoprene, nitrile, Primaloft®, Thinsulate®, alpaca fleece, Polartec® alpha, aerogel composite materials.

Another general aspect of the invention is an open-end AVA cooling mitten able to provide efficient cooling to at least one AVA zone of a front side of a wrist and a palm of a hand. The open-end AVA cooling mitten includes: an insulating layer configured to form a cooling element pocket, the cooling element pocket configured to receive a cooling element configured to extend only over the front side of a wrist and a palm of a hand; a first outer layer in thermally-conductive surrounding relationship with the insulating layer; and a second outer layer attached to the first outer layer so as to form an open-end hand pocket 202 configured to extend only over the wrist and the palm of the hand, the open-end hand pocket configured to allow a plurality of fingers to extend beyond the second outer layer, the open-end hand pocket having an upper inside surface and a lower inside surface, the lower inside surface configured to make thermally-conductive contact with AVA zones of only the front side of the wrist and the palm received by the open-end hand pocket.

In some embodiments, the insulation layer is configured to modulate thermal flow from the cooling element so as to maintain the AVA zones of the palm, and the front side of the wrist at a temperature between 45° F. and 59° F.

In some embodiments, the first outer layer is configured to ensure that the temperature of an AVA zone on a forehead remains within an optimal AVA range of 45° F. to 59° F. when the surface of the first outer layer is pressed against the forehead.

In some embodiments, the cooling element is shaped and sized to be in thermally conducting relationship within the cooling element pocket.

### BRIEF DESCRIPTION OF THE DRAWINGS

Many additional features and advantages will become apparent to those skilled in the art upon reading the following description, when considered in conjunction with the accompanying drawings, wherein:

FIG. 1 is a bottom view of an embodiment of the AVA cooling mitten, showing the opening of the insulated cooling element pocket, and showing the surface of the cooling mitten that can be placed on one's face to cool the AVA zones on the face (cheeks and forehead) while also cooling the AVA zones on the bottom of one's wrists and hands (palm, fingers, and fingertips).

FIG. 2 is a top view of the embodiment of FIG. 2, showing the opening of the hand pocket, and showing a buckle (or a clip) for attaching the mitten to a belt, a harness, a backpack, a sock, or a shoulder strap, for example.

FIG. 3 is a cross-sectional side view of the embodiment of FIGS. 1 and 2, showing the opening of the hand pocket and the opening of the insulated cooling element pocket, also showing the layers that form the hand pocket and the insulated cooling element pocket.

FIG. 4 is a schematic cross-sectional side view corresponding to FIG. 3, showing the layers that form the hand pocket and the insulated cooling element pocket.

FIG. 5 is a bottom view of the embodiment of FIG. 1, also showing a cooling element being inserted into the embodiment of the AVA cooling mitten of FIG. 1.

FIG. 6 is a bottom view of an alternate embodiment of the AVA cooling mitten having a foreshortened closed-end insulated cooling element pocket that receives a short version of a cooling element, the short version substantially cooling the AVA zones of the palm and the bottom of the wrist, but not cooling at least four fingers that extend beyond a commensurately foreshortened open-end hand pocket.

### DETAILED DESCRIPTION

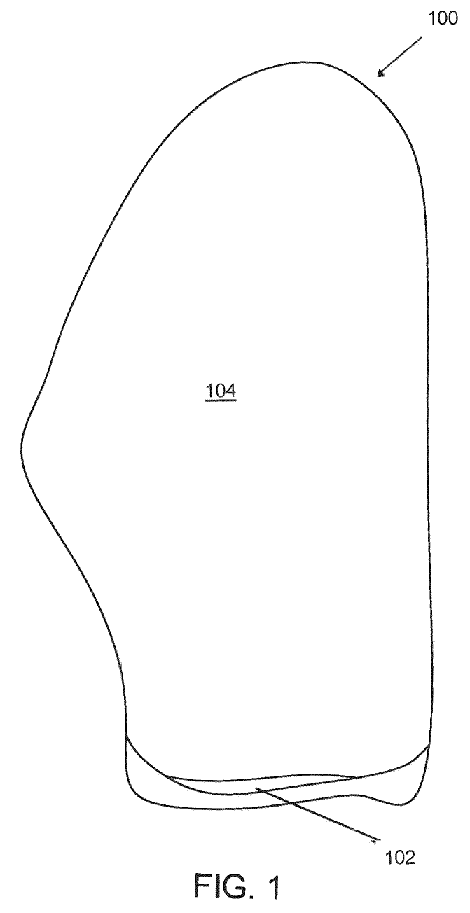

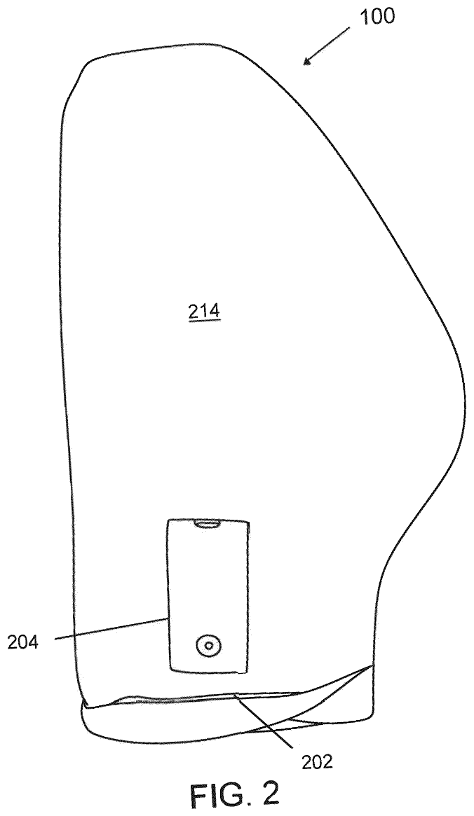

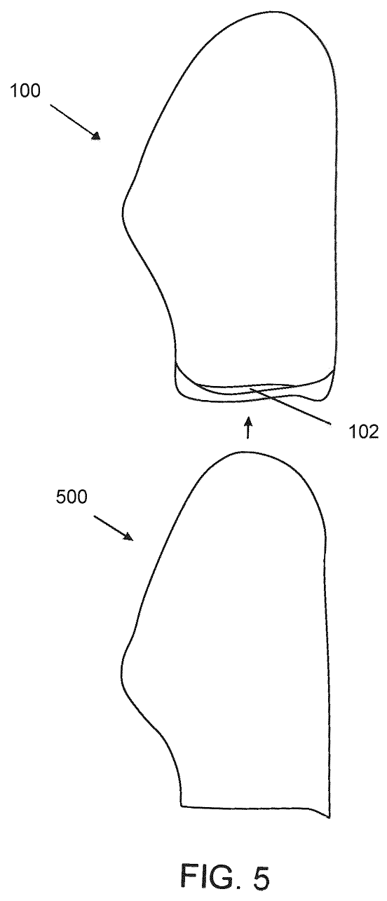

With reference to FIG. 1, an embodiment of the AVA cooling mitten 100 has two pockets: an insulated cooling element pocket 102, and a hand pocket 202 (FIG. 2). The insulated cooling element pocket 102 receives a cooling element 502 (FIG. 5).

The hand pocket 202 is essentially a mitten, in that the hand pocket 202 surrounds all the fingers, including the thumb, in one enclosing pocket 202. The hand pocket can also be configured to include the wrist associated with the hand. The defining characteristic of a mitten is that it doesn't provide individual openings for each finger, unlike a glove, which has separate sections for each digit. While many mittens do have a separate enclosure for the thumb to allow for better grip and dexterity, the absence of individual finger compartments is what fundamentally distinguishes a mitten from a glove. So, even without a separate thumb enclosure, a single-pocket hand covering would still fall within the “mitten” category. Accordingly, another embodiment of the AVA cooling mitten includes a hand pocket 202 that includes a separate thumb pocket (not shown).

The AVA cooling mitten 100 also has a surface 104 of the cooling mitten 100 that can be placed on one's face to cool the AVA zones on the face (cheeks and forehead). Placing the surface 104 on one's face can be done while also cooling the AVA zones on the bottom of one's wrist and the bottom of one's hand (palm, fingers, and fingertips) that has been inserted into the hand pocket 202.

Referring to FIG. 2, the mitten 100 also has a buckle (or a clip) 204 for attaching the mitten 100 to a belt, a harness, a backpack, a sock, or a shoulder strap, for example.

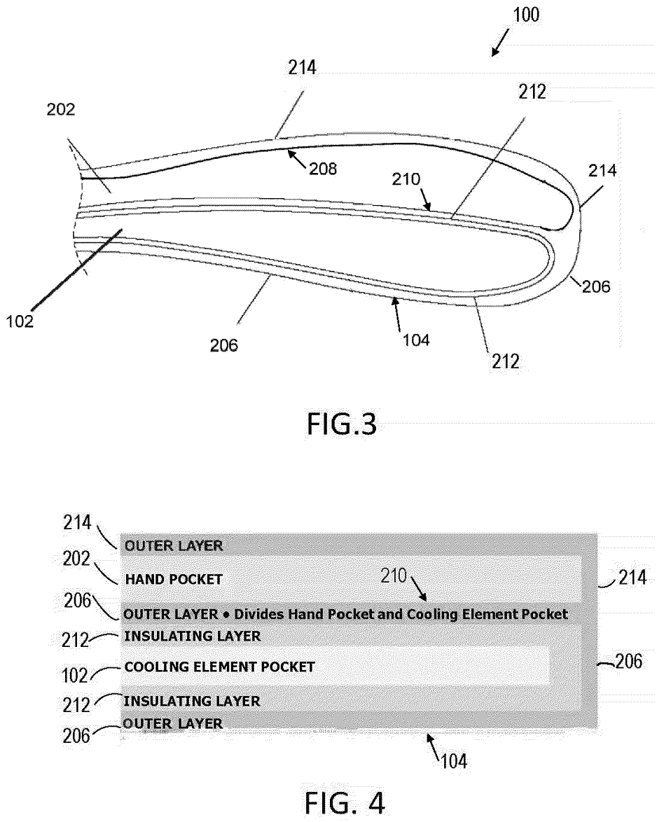

With reference to FIG. 3, a cross-sectional side view of the mitten 100 shows the insulated cooling element pocket 102 and the hand pocket 202. The insulated cooling element pocket 102 is formed from the insulating layer 212 that is sized and shaped to receive a cooling element 500 (FIG. 5). The insulating layer 212 is covered by a first outer layer 206. The hand pocket is formed by attaching the second outer layer 214 so as to form the hand pocket 202.

By inserting one's hand palm down into the hand pocket 202 so as to make thermally conductive contact with the lower inside surface 210 of the hand pocket 202, one can optimally cool the AVA zones on the bottom of the wrist and the hand. Heat from the AVA zones of the hand is thermally conducted through the lower inside surface 210 of the first outer layer 206, and then through the insulating layer 212 to be absorbed by the cooling element 500.

Further, by moving the mitten 100 so that the surface 104 of the first outer layer 206 is pressed against the AVA zones of the cheeks and/or the forehead, the same cooling element 500 that can cool the AVA zones of a hand can also cool the AVA zones of the face. Heat is thermally conducted from the AVA zones of the face through the surface 104 of the first outer layer 206, and then through the insulating layer 212 to be absorbed by the cooling element 500.

Additionally, by moving the mitten 100 so that the surface 104 of the first outer layer 206 is pressed against the AVA zones of the feet, the same cooling element 500 that can cool the AVA zones of a hand can also cool the AVA zones of the feet. Heat is thermally conducted from the AVA zones of the feet through the surface 104 of the first outer layer 206, and then through the insulating layer 212 to be absorbed by the cooling element 500.

Referring to FIG. 4, a schematic cross-sectional side view of the layers 206, 212, and 214 of FIG. 3 is presented to more generally depict the topological relationships (continuity and connectedness) among the layers: 206, 212, and 214. Note that the insulating layer 212 forms the cooling element pocket 102 that is sized and shaped to receive a cooling element 500. Note also that the first outer layer 206 together with the second outer layer 214 form the hand pocket that is sized and shaped to receive a hand and wrist.

The lower inside surface 210 of the hand pocket 202 can optimally cool the AVA zones on the bottom of a wrist and the bottom of a hand inserted into the hand pocket 202.

Also, the surface 104 of the first outer layer 206 of the mitten 100 can optimally cool the AVA zones on the AVA zones of the face, i.e., the cheeks and/or the forehead, when the surface 104 of the first outer layer 206 of the mitten 100 is pressed against the AVA zones of the face.

With reference to FIG. 5, a cooling element 500 is shown being inserted into the insulated cooling element pocket 102 of the AVA cooling mitten 100. The cooling element 500 can be re-usable or single-use. If re-usable, the cooling element 500 can be made using carboxymethyl cellulose (CMC) or sodium polyacrylate (SAP). If single-use, the cooling element 500 can be made using water, ammonium nitrate (NH₄NO₃), calcium ammonium nitrate (can), urea, sodium thiosulfate pentahydrate (Na₂S₂O₃·5H₂O), and a clay-based mixture.

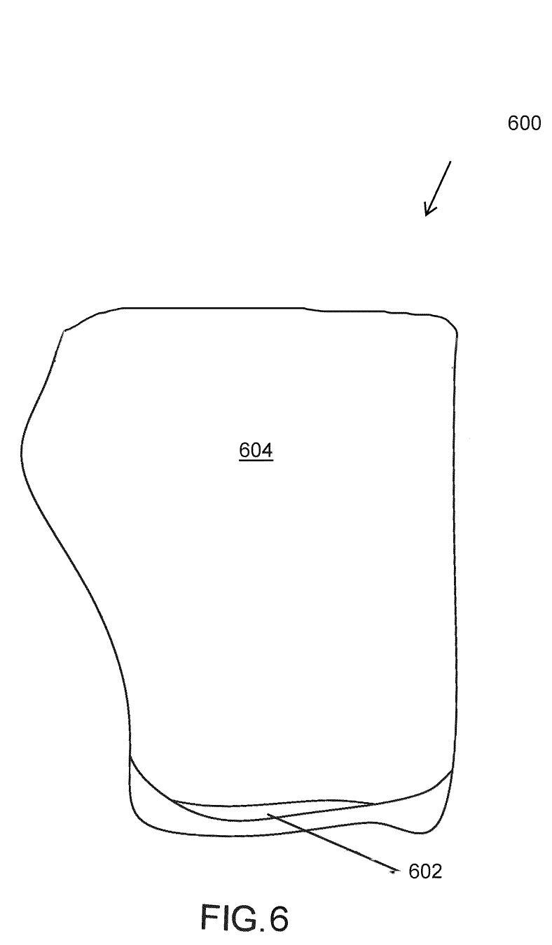

Referring to FIG. 6, an alternate embodiment 600 of an AVA cooling mitten having a foreshortened closed-end insulated cooling element pocket 602 that receives a short version 604 of the cooling element 500, the short version 604 substantially cooling the AVA zones of the palm and the bottom of the wrist, but not the four exposed fingers. Note also that the AVA cooling mitten 600 also has a surface 604 that can be placed on one's face to cool the AVA zones on the face (cheeks and forehead), for example.

The alternate embodiment 600 of the AVA cooling mitten also has a commensurately foreshortened open-end hand pocket 603 that allows at least four fingers to be exposed, but not cooled.

This embodiment 600 has the same essential structures of the embodiment 100 with a closed end hand pocket 202.

One of average skill in the art will appreciate that a set of partial or full finger coverings that do not cool the fingers can be attached to the open-end hand pocket 603.

The embodiment 600 can be worn by athletes, such as soccer players, while actively participating in athletic activities. When used with either a reusable or non-reusable cooling pack, the embodiment 600 will help to lower core body temperature to within the optimal temperature range of 45° F. to 59° F., while engaging in the athletic activity.

The AVA Cooling Mitten of the invention optimizes thermoregulation of the Arterio-Venous Anasto-moses (AVAs) zones to lower core body temperature by independently or simultaneously cooling AVA zones.

Research has identified three AVA zones on a human body, which include the bottom of hands, including the palm, fingers and wrist; the face, including the cheeks and forehead; and the bottoms of the feet. The “optimal AVA temperature range,” to induce heat loss/thermoregulation/cooling of core body temperature is between 45° F. and 59° F. (optimal for thermal conductivity via the AVA zones). Temperatures below this range may cause capillary constriction, and temperatures above this range may not be effective at cooling core body temperatures.

The AVA cooling mitten of the invention provides at least partial coverage of the AVA zones of the bottom of hands, or face, or bottoms of feet, using an insulated textile layer configured to provide an optimal AVA temperature range, a cooling element selected to provide high heat capacity to maintain cold/cool temperatures for a specified period of time, while also being affordable, portable, and easy to use.

The AVA cooling mitten can reduce core body temperature by optimal thermal transfer via at least one AVA zone to increase muscle stamina, muscle recovery, as well as to aid in thermoregulation of the body, such as helping to reduce fever, cool feet for sleeping, and cool down core body temperature to mitigate or prevent heat stroke (hyperthermia).

The embodiments of the AVA cooling mitten address the goal of core body temperature regulation by providing optimal thermal conduction to at least one AVA zone, such as two AVA zones in sequence or simultaneously, i.e. the bottom of hands, the face, or the bottom of a foot.

The AVA cooling mitten employs a mitten-shaped cooling element to provide targeted cooling to entirely or partially cool one or more of the AVA zones, e.g., the bottom of a hand (palm, fingers, wrist), the face (cheeks and forehead), and/or the bottom of a foot.

The AVA cooling mitten 100 includes an insulating layer that ensures the surface temperature of the AVA zones remain within an optimal AVA temperature range of 45° F. and 59° F.

This research indicates an optimal cooling/heating temperature range for AVA zones between 45° and 59° F. Therefore, the insulating layers 304 and 305, of the AVA cooling mitten 001 are configured to transmit cooling/heating temperatures within this optimal AVA cooling range.

The insulating layer 212 and cooling element 500 work together to thermally conduct optimal AVA temperature ranges to the face, bottom of feet, or bottom of hands. These AVA zones can be used sequentially or simultaneously to transmit optimal AVA temperatures to the face and/or bottom of hand so as to reduce core body temperature.

In another embodiment of the AVA cooling mitten 100, the insulating layer 212 can include a material with high heat capacity and low thermal conductivity, such as crushed basalt stone or soapstone.

The AVA cooling mitten 100 provides cooling by carefully regulating the insulating layer 212 between a cooling element 500, to an optimal AVA cooling range of 45° F. and 59° F. The insulating layers of 212 can be configured to transmit optimal temperatures to the AVA zones.

According to this embodiment, more than one insulating layer can be added so as to either increase or decrease the transmitted temperatures to various AVA zones. The insulation layer 212 surrounding the cooling element 500, can be a single, double, or triple layer, for example.

The following are examples of materials used to fabricate the insulating layers to transmit the optimal AVA cooling temperatures. The invention is not limited to these materials nor thickness recommendations:

Thinsulate®: The required thickness is reduced to between 5.66 mm and 9.16 mm. Wool: The required thickness is reduced to between 5.11 mm and 8.28 mm.

Argyle: Similar to wool, the thickness is reduced to between 5.11 mm and 8.28 mm.

Fleece: The thickness is reduced to between 4.56 mm and 7.41 mm. Microfiber: Similar to fleece, the thickness is reduced to between 4.56 mm and 7.41 mm.

Acrylic: The required thickness is reduced but still substantial, between 44.48 mm and 59.97 mm.

Polyester: The thickness is reduced to between 5.11 mm and 8.28 mm.

Nylon: The thickness is reduced but remains impractical, between 56.98 mm and 75.57 mm.

PrimaLoft: The thickness is reduced to between 5.11 mm and 8.28 mm.

Recommended insulators include: Fleece, Microfiber, Thinsulate®, Wool, Polyester, and PrimaLoft®. Each of these insulating materials offer the best performance, requiring thicknesses in the range of approximately 4.5 to 9.2 mm. Other potential insulators include: Acrylic, Nylon, and spandex. Each of these insulating materials may require impractical thicknesses.

The insulation layer 212 textile can be made from: recycled polyester, neoprene, nitrile, Primaloft®, Thinsulate®, alpaca fleece, Polartec® alpha, aerogel composite materials, for example.

The AVA cooling mitten 100 can include a reusable and/or a non-reusable cooling element 500.

The AVA cooling mitten is configured to allow a hand to be inserted while in an “open hand” position (i.e., not closed, not a fist, not gripping) to facilitate cooling. An open hand position allows for maximum capillary blood flow and core temperature cooling effects.

The AVA cooling mitten 100 can simultaneously cool AVA zones on the bottom of the hands and/or AVA zones on the bottom of feet and/or the AVA zones on the face. Simultaneous AVA zone cooling can be performed by placing the AVA cooling mitten 100 on the intended AVA zone, such as the bottom of a foot, or the face, even with a hand inserted in the AVA cooling mitten 100.

A pair of AVA cooling mittens 100 can be worn simultaneously, one on each hand, or a single AVA colling mitten 100 can be worn on each hand sequentially.

An AVA cooling mitten 100 can be used intermittently, such as between exercises to facilitate a cooling off break, or can be used continuously during exercise, or after exercise, or during exposure to warm temperatures.

The AVA cooling mitten 100 can include a fastening or closure mechanism to fasten or tighten the mitten 100 around a wrist (not shown).

Some embodiments of the AVA cooling mitten 100 can have an icon or a tab at or near the opening of each pocket 102, 202 to indicate the purpose of each pocket.

The opening of the cooling element pocket 102 may be smaller or elastic or use other means to keep the cooling element 500 secure inside 102.

Each cooling element 500 is generally flat, and can have dimensions of 8″×4″ with a ½″ thickness, or 1″ thickness, for example. The cooling element 500 is flat so it can make effective thermally conductive contact with the AVA zones of an open hand. Further, it is best to insert one's hand in an “open palm position”, which is vital for proper blood flow in the hand. Contracting the hand, or making a fist, limits blood flow, and therefore the flat cooling element is optimal cooling of the bottom of the AVA zones of an open hand.

Prior to each use of the AVA cooling mitten 100, the AVA cooling mitten 100 with a reusable cooling element 500 inserted in the insulated cooling element pocket 102 can be stored inside a freezer or other cold location (e.g., a cooler or an ice chest) when not in use. Once the reusable cooling element 500 is frozen, the AVA cooling mitten 100 with the reusable cooling element 500 is removed from the cold location, and the AVA cooling mitten 100 is ready to use. A hand can then be promptly inserted into the hand pocket 202 of the AVA cooling mitten 100, resulting in immediate AVA cooling within the optimal AVA temperature range of the AVA areas of the hand.

While the AVA cooling mitten 100 is worn on the hand, the AVA cooling mitten 100 can be placed on a second AVA zone, such as an AVA zone of either the face or the bottom of a foot to achieve simultaneous optimal cooling of two major AVA zones, thereby maximizing the rate of reduction of the core body temperature.

The reusable cooling element 500 can also be stored separately from the mitten, in a freezer or other cold location when not in use. Then, once frozen, the reusable cooling element 500 can be inserted inside the insulated cooling element pocket 102, making the AVA cooling mitten 100 ready to use as described above.

Additionally, a user can have a spare frozen cooling element 500, and can switch out a used cooling element 500 with the spare frozen cooling element 500, as soon as the surface temperature of the original cooling element 500 exceeds the optimal AVA cooling temperature range.

The mitten-shaped cooling element 500 can be implemented as a one-time use (non-reusable) instant ice pack that is formulated for sustained cooling at optimal temperatures. This is especially practical in cases where the temperature of a reusable cooling element 500 rises above optimal AVA cooling values, but a frozen re-usable replacement is not available.

Also, in conditions or environments where refrigeration is not available, such for in certain military, camping, or other outdoor environments without electricity, a non-reusable cooling element 500 is essential. For another example, a situation where a firefighter is experiencing elevated core body temperature from working within a burning building, the firefighter can quickly and easily don an AVA colling mitten 100 with an instant non-reusable cooling element 500, thereby preventing heat stroke.

The AVA cooling mitten 100 can be stored at room temperature when not in use. An instant cold pack 500 can be activated by shaking, twisting, or otherwise following instructions to activate the cooling reaction, and then inserted into the insulated cooling element pocket 102. The AVA cooling mitten 100 would be activated and ready to use as described above. Or, the non-reusable heating/cooling element 500 can be activated while inside the insulated cooling element pocket 102.

A reusable cooling element 500, such as a gel-pack, can be made from carboxymethyl cellulose (CMC), sodium polyacrylate (SAP), or other cooling compositions or formulas that are made for extended cooling duration.

A non-reusable cooling element 500 can be made from water, ammonium nitrate (NH₄NO₃), calcium ammonium, nitrate (can), urea, sodium thiosulfate pentahydrate (Na₂S₂O₃·5H₂O), clay-based mixtures, or other cooling/heating compositions or formulas that are made for extended cooling/heating duration.

To use the AVA cooling mitten 100, the user simply inserts a hand into the hand pocket 202. The opening of the hand pocket 202 can be smaller than the hand, the opening having an elastic band sewn in, or the opening can incorporate hook and loop material for securing the hand inside the hand pocket 202.

Thus, the AVA cooling mitten 100 is easy to use by simply slipping a hand inside the hand pocket 202. An elastic band in the opening of the hand pocket 202 can be expanded and then allowed to contract so as to keep the hand secure inside the hand pocket 202. Or, the hook and loop material closure can be opened to allow the hand inside the hand pocket 202, and then be secured inside the hand pocket 202.

One of the most important features of the AVA cooling mitten 100 is that it can be easily worn by any person, regardless of age, mental health condition, or circumstance. For everyday use cases, such as when used by a weightlifter, an athlete, or an outdoor worker, the AVA cooling mitten 100 provides easy access to repetitive AVA cooling to help regulate core body temperature.

For those exposed to extreme heat, like a firefighter or an athlete or an outdoor worker going into heat stroke, ease-of-use is imperative to quickly lower core body temperature. This is especially true when the AVA cooling mitten 100 is used on the cheeks and forehead, and therefore serves to simultaneously cool all five known AVA areas of the bottom of hands and face (palms, fingers, wrists, cheeks, and forehead). Multiple simultaneous AVA zone cooling is a unique feature of 100, and can provide innumerable benefits during everyday use, as well as in emergency situations.

Ease-of-Use is especially important for the elderly, those with impaired vision, and anyone that needs the product to work quickly and easily, such as a firefighter after extreme heat exposure, or an athlete or outdoor worker on the verge of collapse from heat stroke. A person struggling with heat stroke can be mentally impaired, and so “ease-of-use” is imperative.

In another embodiment, the AVA cooling mitten 100 can include a buckle 204 or a fastener to attach the AVA cooling mitten 100 to a belt, a harness, a shoulder strap, a waistband, or a backpack, for example, to ensure that the AVA cooling mitten 100 is readily available.

While the present Detailed Description describes particular illustrative embodiments, the invention is not limited to these embodiment. Therefore, the description of the embodiments provided herein is illustrative of many possible embodiments of the present invention, and does not limit the scope of the invention as claimed. Further, while the Detailed Description describes an embodiment of an AVA cooling mitten, it will be appreciated that the invention can be extended to other articles of clothing that employ AVA cooling, such as a headband, a cooling mask, an earmuff, a cooling wrap, a boot, a sock, a shoe, a shoe insert, a glove, and a fingerless glove, for example.

What is claimed is:

1. An AVA cooling mitten able to provide efficient cooling to at least one AVA zone of a front side of a wrist and a front side of a hand having a palm and fingers, the AVA cooling mitten comprising:
an insulating layer configured to form a cooling element pocket, the cooling element pocket configured to receive a cooling element;
a first outer layer in thermally-conductive surrounding relationship with the insulating layer; and
a second outer layer attached to the first outer layer so as to form a hand pocket, the hand pocket configured to receive and conform to a hand and a wrist, the hand pocket having an upper inside surface and a lower inside surface, the lower inside surface configured to make thermally-conductive contact with AVA zones of the front side of the wrist and the front side of the hand received by the hand pocket.

2. The AVA cooling mitten of claim 1, wherein the insulation layer is configured to modulate thermal flow from the cooling element so as to maintain the AVA zones of the palm, and the front side of the wrist and fingers at a temperature between 45° F. and 59° F.

3. The AVA cooling mitten of claim 1, wherein the first outer layer is configured to ensure that the temperature of an AVA zone on a forehead remains within an optimal AVA range of 45° F. to 59° F. when the surface of the first outer layer is pressed against the forehead.

4. The AVA cooling mitten of claim 1, wherein the cooling element is shaped and sized to be in thermally conducting relationship within the cooling element pocket.

5. The AVA cooling mitten claim of 1, wherein the cooling element is a re-usable cold pack, and is configured to be frozen before being inserted into the cooling element pocket, and to be removed from the cooling element pocket after the re-usable cold pack melts.

6. The AVA cooling mitten claim of 1, wherein the cooling element is a non-reusable instant ice-pack that is ready-to-use once inserted into the cooling element pocket.

7. The AVA cooling mitten of claim 1, further comprising:
an attachment device connected to the second outer layer, the attachment device configured to attach to one of: a belt, a strap, a harness, a sock, or a backpack.

8. The AVA cooling mitten of claim 7, wherein the attachment device is one of: a buckle, a snap, or hook and loop material.

9. The AVA cooling mitten of claim 5, wherein the re-usable cooling element includes:
carboxymethyl cellulose (CMC) or sodium polyacrylate (SAP).

10. The AVA cooling mitten of claim 6, wherein the non-reusable cooling element includes:
water, ammonium nitrate (NH₄NO₃), calcium ammonium, nitrate (can), urea, sodium thiosulfate pentahydrate (Na₂S₂O₃·5H₂O), clay-based mixture.

11. The AVA cooling mitten of claim 1, wherein the insulation layer is made using at least one of:
fleece, wool, recycled polyester insulation, down, merino wool, aerogel, alpaca fleece.

12. The AVA cooling mitten of claim 1, wherein the first outer layer is made using at least one of:
recycled polyester, neoprene, nitrile, Primaloft®, Thinsulate®, alpaca fleece, Polartec® alpha, aerogel composite materials.

13. The AVA cooling mitten of claim 1 wherein the second outer layer is made using at least one of:
recycled polyester, neoprene, nitrile, Primaloft®, Thinsulate®, alpaca fleece, Polartec® alpha, aerogel composite materials.

14. An open-end AVA cooling mitten able to provide efficient cooling to at least one AVA zone of a front side of a wrist and a palm of a hand, the open-end AVA cooling mitten comprising:
an insulating layer configured to form a cooling element pocket, the cooling element pocket configured to receive a cooling element configured to extend only over the front side of a wrist and a palm of a hand;
a first outer layer in thermally-conductive surrounding relationship with the insulating layer; and
a second outer layer attached to the first outer layer so as to form an open-end hand pocket 202 configured to extend only over the wrist and the palm of the hand, the open-end hand pocket configured to allow a plurality of fingers to extend beyond the second outer layer, the open-end hand pocket having an upper inside surface and a lower inside surface, the lower inside surface configured to make thermally-conductive contact with AVA zones of only the front side of the wrist and the palm received by the open-end hand pocket.

15. The open-end AVA cooling mitten of claim 14, wherein the insulation layer is configured to modulate thermal flow from the cooling element so as to maintain the AVA zones of the palm, and the front side of the wrist at a temperature between 45° F. and 59° F.

16. The open-end AVA cooling mitten of claim 14, wherein the first outer layer is configured to ensure that the temperature of an AVA zone on a forehead remains within an optimal AVA range of 45° F. to 59° F. when the surface of the first outer layer is pressed against the forehead.

17. The open-end AVA cooling mitten of claim 14, wherein the cooling element is shaped and sized to be in thermally conducting relationship within the cooling element pocket.
